(19) United States
(12) Patent Application Publication
(10) Pub. No.: US 2015/0335314 A1
(43) Pub. Date: Nov. 26, 2015

(54) ULTRASONIC DIAGNOSTIC DEVICE

(71) Applicant: HITACHI ALOKA MEDICAL, LTD., Mitaka-shi, Tokyo (JP)

(72) Inventor: Kazuhiro Amino, Mitaka-shi (JP)

(73) Assignee: HITACHI ALOKA MEDICAL, LTD., Mitaka-shi, Tokyo (JP)

(21) Appl. No.: 14/759,357

(22) PCT Filed: Dec. 25, 2013
(86) PCT No.: PCT/JP2013/084604

§ 371 Date: Jul. 6, 2015

(30) Foreign Application Priority Data
Jan. 8, 2013 (JP) 2013-001126

(51) Int. Cl. A61B 8/08; G01S 7/52; G01S 15/89

(52) U.S. Cl. CPC A61B 8/488; G01S 15/8979; G01S 7/52085; A61B 8/5207

(57) ABSTRACT

A received signal is obtained along an ultrasonic beam while the ultrasonic beam which passes through the measurement area is moved in periodic fashion inside the measurement area. A sampling processing unit (20) uses a plurality of sampling sets mutually offset in time phase, and samples received signals across a plurality of time phases for each sampling set to thereby obtain a received data string for each sampling set. Doppler information in the measurement area is then obtained on the basis of the plurality of received data strings that correspond to the plurality of sampling sets.

### TECHNICAL FIELD

The present invention relates to an ultrasonic diagnostic device, and more particularly to a technique for obtaining Doppler information.

### BACKGROUND ART

There have been known techniques of using a continuous wave or a pulse wave in an ultrasonic diagnostic device to obtain Doppler information from blood flow or the like in a living body (Patent Documents 1 and 2). In measuring high velocities, continuous-wave Doppler using a continuous wave is more advantageous over pulsed Doppler using a pulse wave. On the other hand, pulsed Doppler enables determination of location information in addition to Doppler information concerning velocity or the like.

In performing Doppler measurement using an ultrasonic diagnostic device, a cursor or the like is set by a user (testing personnel) within an ultrasonic image, and the location of this cursor serves as the target of the Doppler measurement. However, when, for example, blood flow within a heart is to be measured, even when the location of the cursor is set to match the location of the blood flow that should be the measurement target, there are cases in which the location of the cursor becomes shifted from the location of the blood flow, due to the movement of the heart itself or the body movement of the entire living body. In such cases, for example, velocity or the like of the blood flow over one heartbeat cannot be measured stably.

### PRIOR ART LITERATURE

### Patent Documents

Patent Document 1: JP 2002-301077 A

Patent Document 2: JP 2000-175915 A

### SUMMARY OF THE INVENTION

### Problems to be Solved by the Invention

In light of the above-described background art, the present inventors have conducted research and development regarding Doppler measurement in an ultrasonic diagnostic device. In particular, the present inventors have focused on a technique for increasing reliability of Doppler information.

The present invention was created the course of during such research and development. An object of the present invention is to provide an improvement technique that increases reliability of Doppler information in an ultrasonic diagnostic device.

### Means for Solving the Problems

An ultrasonic (ultrasound) diagnostic device that achieves the above-noted object comprises a probe that transmits and receives ultrasonic waves; a transmission-reception control unit that controls the probe to thereby acquire an ultrasonic reception signal while moving an ultrasonic beam in a periodic manner; a sampling processing unit that employs a plurality of sampling sets that are shifted in time phase from each other, and performs a sampling process on the reception signal over a plurality of time phases for each of the sampling sets, so as to obtain a reception data string for each of the sampling sets; and a Doppler processing unit that obtains Doppler information based on a plurality of reception data strings corresponding to the plurality of sampling sets.

The probe in the above-described preferred ultrasonic diagnostic device is preferably a sector-scan type ultrasonic probe in which an angle of a continuous-wave ultrasonic beam (transmission beam and reception beam) can be changed electronically within a tomographic plane. Naturally, the probe may be an ultrasonic probe of other scan types, such as a linear-scan type ultrasonic probe that moves an ultrasonic beam substantially in parallel, or a three-dimensional ultrasonic probe that can scan an ultrasonic beam three-dimensionally. In obtaining the Doppler information, pulsed Doppler using a pulse wave may alternatively be employed, instead of continuous-wave Doppler using a continuous wave.

The ultrasonic beam is moved within a measurement area, for example. The measurement area is an area containing a measurement target (for example, a blood flow, a moving tissue, etc.). The above-described preferred ultrasonic diagnostic device moves the ultrasonic beam in periodic manner within the measurement area, and, while doing so, acquires the reception signal along the ultrasonic beam. Incases in which the measurement target moves, an area covering the range of movement of the measurement target is desirably set as the measurement area.

In the above-described preferred ultrasonic diagnostic device, the Doppler information is obtained from the reception signal acquired along the ultrasonic beam while moving the ultrasonic beam in a periodic manner within the measurement area, for example. With this arrangement, even in cases in which the measurement target moves within the measurement area, the Doppler information of the measurement target can be obtained while acquiring the reception signal from the measurement area such that the range of movement of the measurement target is covered. In this way, reliability of the Doppler information can be enhanced.

In a preferred embodiment, the transmission-reception control unit moves the ultrasonic beam in a periodic manner so as to acquire the reception signal repeatedly at a plurality of measurement lines passing through the measurement area, for example. Further, the sampling processing unit employs the plurality of sampling sets corresponding to the plurality of measurement lines, and obtains, for each sampling set, a reception data string regarding its corresponding measurement line, thereby obtaining a plurality of reception data strings corresponding to the plurality of measurement lines.

In a preferred embodiment, the transmission-reception control unit employs a beam cursor set according to a user operation as a reference measurement line, sets an additional measurement line near the reference measurement line, and moves the ultrasonic beam in a periodic manner so as to acquire the reception signal repeatedly at the reference measurement line and the additional measurement line.

In a preferred embodiment, the Doppler processing unit analyzes a Doppler shift amount concerning each of the reception data strings, and generates a Doppler waveform based on a plurality of Doppler shift amounts obtained from the plurality of reception data strings.

In a preferred embodiment, the Doppler processing unit selects a maximum Doppler shift amount for each time point from among the plurality of reception data strings, and generates a Doppler waveform showing, across a plurality of time points, maximum Doppler shift amounts corresponding to respective ones of the plurality of time points.

### Advantages of the Invention

The present invention provides an improvement technique that increases reliability of Doppler information in an ultrasonic diagnostic device. For example, in a preferred ultrasonic diagnostic device according to the present invention, even in cases in which a measurement target moves, Doppler information of the measurement target can be obtained while acquiring a reception signal from a measurement area such that the range of movement of the measurement target is covered.

### BRIEF DESCRIPTION OF THE DRAWINGS

FIG. 1 is a block diagram showing an overall configuration of a preferred ultrasonic diagnostic device according to the present invention.

FIG. 2 is a diagram for explaining setting of a direction and a focus point of an ultrasonic beam.

FIG. 3. is a diagram for explaining setting of a plurality of measurement lines.

FIG. 4 is a diagram for explaining a sampling process performed on a reception beam signal.

FIG. 5 is a diagram for explaining a Doppler waveform generating process.

### EMBODIMENTS OF THE INVENTION

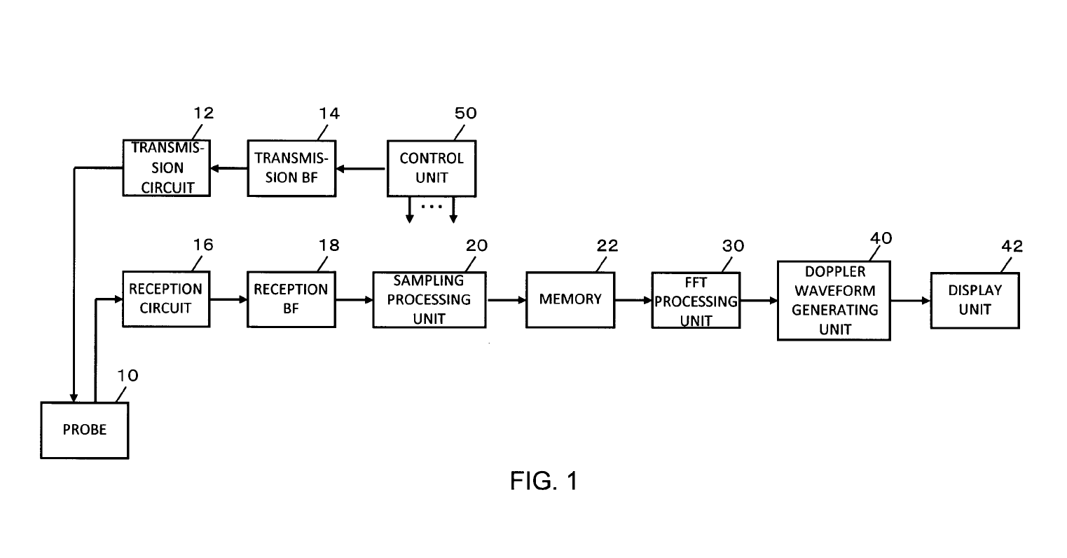

FIG. 1 is a block diagram showing an overall configuration of an ultrasonic diagnostic device that is preferred for practicing the present invention. The ultrasonic diagnostic device of FIG. 1 is capable of transmitting and receiving continuous ultrasonic waves to and from a measurement target such as a blood flow or a moving tissue, and obtaining Doppler information from the measurement target.

A probe 10 is an ultrasonic probe that transmits and receives continuous ultrasonic waves. The probe 10 includes a plurality of oscillating elements which may be arrayed one-dimensionally (i.e., linearly). For example, those oscillation elements located toward one side from the center of the array are transmitting elements, and those oscillation elements located toward the other side are receiving elements. Ultrasonic waves are continuously transmitted by the plurality of transmitting oscillation elements, and ultrasonic waves are continuously received by the plurality of receiving oscillation elements.

The transmitting oscillation elements provided in the probe 10 transmit continuous ultrasonic waves based on transmission signals output from a transmission circuit 12 to the respective oscillation elements. The transmission circuit 12 is controlled by a transmission beam former (transmission BF) 14.

The transmission beam former 14 causes the transmission circuit 12 to output a plurality of transmission signals and controls transmission carried out by the transmitting oscillation elements, to thereby generate and scan a continuous-wave transmission beam.

The receiving oscillation elements provided in the probe 10 receive continuous ultrasonic waves and output the received signals. The plurality of received signals acquired by the plurality of receiving oscillation elements are sent to a reception circuit 16. Each of the received signals is subjected to processing such as quadrature detection and sent to a reception beam former (reception BF) 18.

The reception beam former 18 forms a reception beam by performing phased addition or the like of the plurality of received signals obtained from the plurality of receiving oscillation elements via the reception circuit 16, so that a reception beam signal is obtained along the reception beam.

Directions and a focus point of the ultrasonic beams (the transmission beam and the reception beam) are set according to an operation by a user (testing personnel).

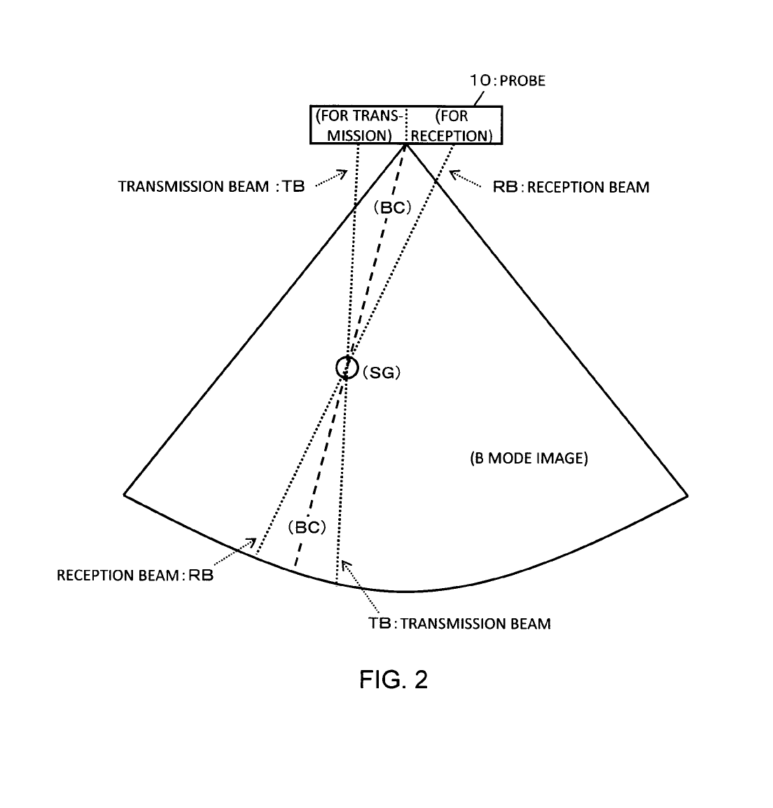

FIG. 2 is a diagram for explaining setting of a direction and a focus point of an ultrasonic beam. In setting these items, a B-mode image containing the measurement target is initially generated. For example, when a blood flow at a heart valve is to be examined, pulsed ultrasonic waves are transmitted and received by the probe 10 within an area containing the heart, and a known process of forming a B-mode image is carried out, so that a B-mode image showing the inside of the heart is displayed on a display unit 42 (FIG. 1).

The user (testing personnel) sets a beam cursor BC while checking in the B-mode image, so that the beam cursor BC passes through the heart valve at which the blood flow to be measured is present. For example, the angle of the beam cursor BC is changeable in the B-mode image, and the user sets the beam cursor BC to a desired angle (i.e., direction) using an operation device.

When the beam cursor BC is set, the user sets, while checking in the B-mode image, a sample gate SG at the location of the blood flow to be measured. For example, the location (i.e., depth) of the sample gate SG is changeable on the beam cursor BC, and the user sets the sample gate SG at a desired location (depth) using an operation device. The size and shape of the sample gate SG may be changeable.

When the sample gate SG is set, a continuous-wave transmission beam TB and a reception beam RB are generated such that those beams pass through the location of the sample gate SG. Further, the transmission beam TB and the reception beam RB are generated with the location of the sample gate SG serving as the focus point. As a result, as shown in FIG. 2, for example, the transmission beam TB of the transmitting oscillation elements of the probe 10 and the reception beam RB of the receiving oscillation elements are set to intersect each other at the location of the sample gate SG.

Referring again to FIG. 1, when the directions and the focus point of the ultrasonic beams (the transmission beam and the reception beam) are set, transmission and reception are performed using continuous ultrasonic waves, and a reception beam signal is obtained along the reception beam in the reception beam former 18.

A sampling processing unit 20 performs sampling and digitization of the reception beam signal so as to obtain reception data strings. The reception data strings are temporarily stored in a memory 22, and then processed in an FFT processing unit (fast Fourier transform processing unit) 30.

The FFT processing unit (fast Fourier transform processing unit) 30 executes fast Fourier transform calculation with respect to the reception data strings so as to perform frequency analysis of the reception data strings. In the FFT processing unit 30, the reception data strings are converted into frequency spectrum data.

A Doppler waveform generating unit 40 generates a Doppler waveform based on Doppler shift amounts (Doppler shift frequencies) obtained from the frequency spectrum data concerning the reception data strings. As a result, a Doppler waveform of the measurement target such as a blood flow at a heart valve can be obtained. The generated Doppler waveform is displayed on the display unit 42.

A control unit 50 controls the overall ultrasonic diagnostic device of FIG. 1. Among the elements (functional blocks) shown in FIG. 1, the transmission circuit 12, the transmission beam former 14, the reception circuit 16, the reception beam former 18, the sampling processing unit 20, the FFT processing unit 30, and the Doppler waveform generating unit 40 are each implemented with hardware such as electric or electronic circuitry and processors, and upon such implementation, devices such as a memory may be used as necessary. Further, a preferred embodiment of the display unit 42 is a liquid crystal display or the like. Furthermore, the control unit 50 can be implemented by, for example, cooperative operation of hardware such as CPU, processors, and memory, and software (program) defining operations of the CPU and processors.

The outline of the ultrasonic diagnostic device of FIG. 1 is as described above. Even incases in which a measurement target moves, the ultrasonic diagnostic device of FIG. 1 is capable of generating a Doppler waveform of the measurement target while acquiring a reception signal from a measurement area such that the range of movement of the measurement target is covered. Processes related to such generation of the Doppler waveform are next described in detail. In the following description, the elements (blocks) shown in FIG. 1 are referred to using the reference numerals used in FIG. 1.

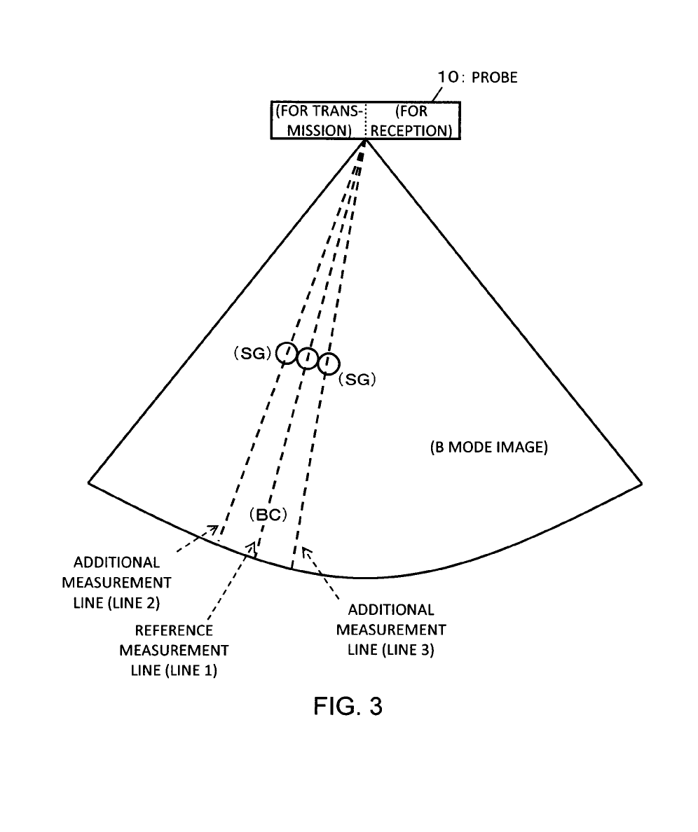

FIG. 3. is a diagram for explaining setting of a plurality of measurement lines. The ultrasonic diagnostic device of FIG. 1 sets a plurality of measurement lines such that the measurement lines pass through a measurement area containing the measurement target, and moves an ultrasonic beam in periodic manner so as to obtain a reception signal repeatedly at the plurality of measurement lines. FIG. 3 shows a specific example of the plurality of measurement lines.

When the beam cursor BC and the sample gate SG located on the beam cursor BC are set (see FIG. 2), the control unit 50 sets the beam cursor BC as a reference measurement line (line 1). Further, the control unit 50 sets additional measurement lines (line 2, line 3) near the reference measurement line (line 1). For example, within the B-mode image, at beam line locations spaced from the reference measurement line (line 1) to the left and the right by designated number of beam lines, a left additional measurement line (line 2) and a right additional measurement line (line 3) are set. The designated number of beam lines may be changeable by the user, or a fixed value may be used as the designated number of beam lines.

Further, the control unit 50 sets sample gates SG on the respective additional measurement lines (line 2, line 3), in locations near the sample gate SG on the reference measurement line (line 1). For example, the sample gates SG are set at the same depth on all of the reference measurement line (line 1) and the additional measurement lines (line 2, line 3).

When the plurality of measurement lines are set, the control unit 50 controls the transmission beam former 14 and the reception beam former 18 such that the reception signal is acquired repeatedly at the measurement lines. For example, first, the continuous-wave transmission beam TB and the reception beam RB are generated such that the beams pass through the location of the sample gate SG on the reference measurement line (line 1), with this location serving as the focus point, and the reception beam signal is obtained along the reception beam RB.

Next, the continuous-wave transmission beam TB and the reception beam RB are generated such that the beams pass through the location of the sample gate SG on the left additional measurement line (line 2), with this location serving as the focus point, and the reception beam signal is obtained along the reception beam RB. Subsequently, the continuous-wave transmission beam TB and the reception beam RB are generated such that the beams pass through the location of the sample gate SG on the right additional measurement line (line 3), with this location serving as the focus point, and the reception beam signal is obtained along the reception beam RB. After that, generation of continuous-wave ultrasonic beams (transmission beam TB and reception beam RB) is performed while returning back to the reference measurement line (line 1).

In this way, for example, the reception beam signal is obtained successively by repeating the process in the order of the reference measurement line (line 1), the additional measurement line (line 2), and the additional measurement line (line 3). The reception beam signal is digitized in the sampling processing unit 20.

The sampling processing unit 20 employs a plurality of sampling sets that are shifted in time phase from each other, and performs a sampling process on the reception beam signal over a plurality of time phases for each of the sampling sets, so as to obtain a reception data string for each sampling set.

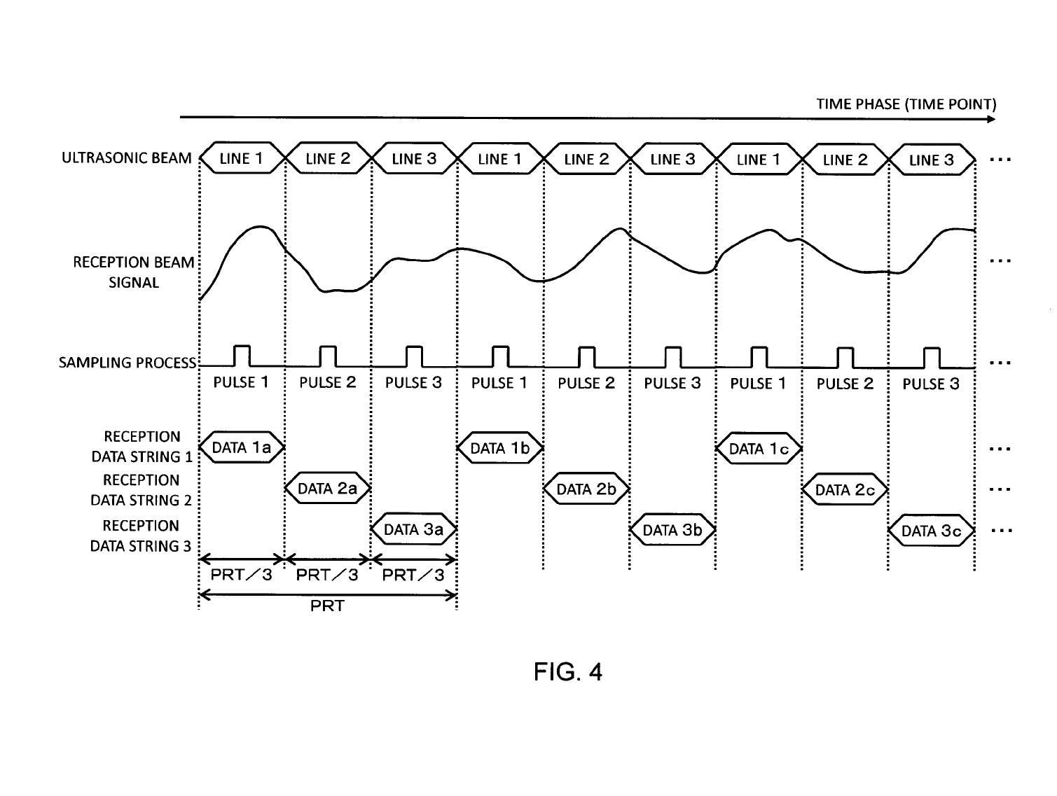

FIG. 4 is a diagram for explaining a sampling process performed on the reception beam signal. The sampling processing unit 20 employs a plurality of sampling sets corresponding to the plurality of measurement lines, and obtains, for each of the sampling sets, a reception data string for the corresponding measurement line, so as to obtain a plurality of reception data strings corresponding to the respective measurement lines.

FIG. 4 illustrates, with the horizontal axis indicating time phase (time point), a process performed up to the point when the plurality of reception data strings are obtained. The ultrasonic beam is controlled to repeat scanning of the reference measurement line (line 1), the additional measurement line (line 2), and the additional measurement line (line 3) in that order, and a reception beam signal is obtained along the ultrasonic beam (see FIG. 3).

When performing the sampling process, the sampling processing unit 20 employs sampling sets corresponding to the respective measurement lines. In FIG. 4, a sampling set comprising a plurality of pulses 1 is correlated to the reference measurement line (line 1). Further, a sampling set comprising a plurality of pulses 2 is correlated to the additional measurement line (line 2), and a sampling set comprising a plurality of pulses 3 is correlated to the additional measurement line (line 3).

The plurality of pulses 1 are correlated to periods of the reference measurement line (line 1) that are repeated periodically in the reception beam signal acquired over a plurality of time phases. The sampling processing unit 20 performs a sampling process (i.e., digitization) on the reception beam signal at the timings of the respective pulses 1, and thereby obtains a reception data string 1 composed of data 1a, data 1b, data 1c, and so on.

Since the plurality of pulses 1 are correlated to the periods of the reference measurement line (line 1), the reception data string 1 is a result obtained by sampling the reception beam signal segments corresponding to the reference measurement line (line 1). In other words, the reception data string 1 is a result obtained by sampling the reception beam signal segments obtained consecutively along the reception beam corresponding to the reference measurement line (line 1).

Further, the plurality of pulses 2 are correlated to periods of the additional measurement line (line 2) that are repeated periodically in the reception beam signal acquired over a plurality of time phases. The sampling processing unit 20 performs a sampling process (i.e., digitization) on the reception beam signal at the timings of the respective pulses 2, and thereby obtains a reception data string 2 composed of data 2a, data 2b, data 2c, and so on.

Since the plurality of pulses 2 are correlated to periods of the additional measurement line (line 2), the reception data string 2 is a result obtained by sampling the reception beam signal segments corresponding to the additional measurement line (line 2). In other words, the reception data string 2 is a result obtained by sampling the reception beam signal segments obtained consecutively along the reception beam corresponding to the additional measurement line (line 2).

Similarly, the plurality of pulses 3 are correlated to periods of the additional measurement line (line 3) that are repeated periodically in the reception beam signal acquired over a plurality of time phases. The sampling processing unit 20 performs a sampling process (i.e., digitization) on the reception beam signal at the timings of the respective pulses 3, and thereby obtains a reception data string 3 composed of data 3a, data 3b, data 3c, and so on.

Since the plurality of pulses 3 are correlated to periods of the additional measurement line (line 3), the reception data string 3 is a result obtained by sampling the reception beam signal segments corresponding to the additional measurement line (line 3). In other words, the reception data string 3 is a result obtained by sampling the reception beam signal segments obtained consecutively along the reception beam corresponding to the additional measurement line (line 3).

A pulse repetition time (PRT), which is a duration of time from pulse 1 to pulse 3 during the sampling process, is determined depending on a flow velocity range. For example, depending on a Doppler-waveform flow velocity range set by the user, in order that the maximum flow velocity (the maximum flow velocity in a positive direction and the maximum flow velocity in a negative direction) of the flow velocity range can be measured without generating a loopback phenomenon, a pulse repetition frequency (PRF) is determined at a value that is double a Doppler shift frequency corresponding to the maximum flow velocity, and the pulse repetition time (given by PRT=1/PRF) is set for the sampling process.

Further, a pulse time for each of pulse 1, pulse 2, and pulse 3 in the sampling process is set to a value that is ⅓ of the pulse repetition time (PRT) (i.e., PRT/3). Here, although the specific embodiment described with reference to FIGS. 3 and 4 relates to a case in which the number of measurement lines is three, the number of measurement lines may alternatively be any plural number other than three. In such cases, in accordance with the number N of measurement lines, N sampling sets are employed, and each pulse time in the sampling process is set to a value that is 1/N of the pulse repetition time (PRT) (i.e., PRT/N).

Referring again to the specific embodiment of FIG. 4, the reception data strings 1-3 obtained in the sampling processing unit 20 are stored in the memory 22. Subsequently, the reception data strings 1-3 stored in the memory 22 are processed in the FFT processing unit (fast Fourier transform processing unit) 30.

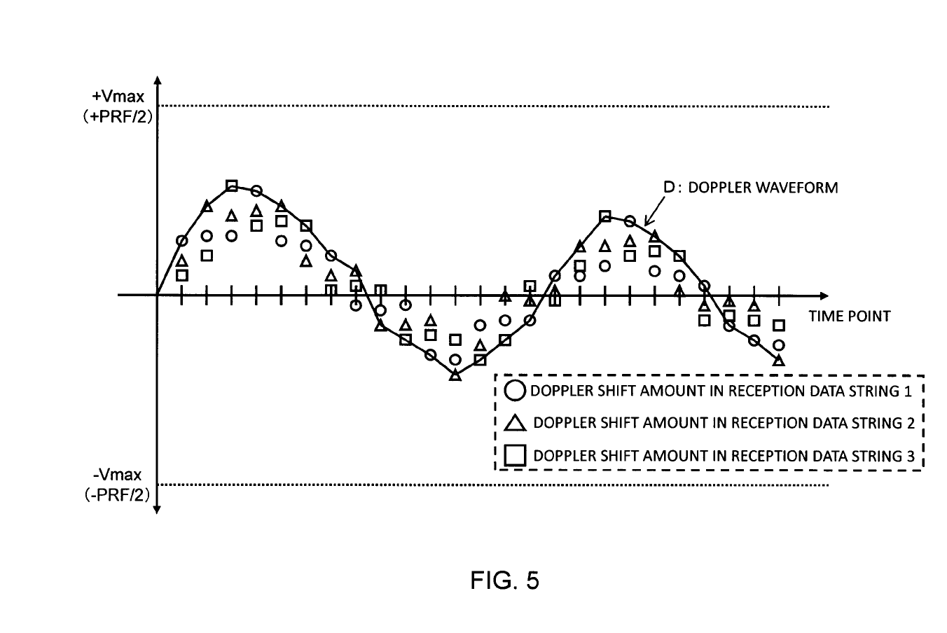

FIG. 5 is a diagram for explaining the Doppler waveform generating process. The FFT processing unit (fast Fourier transform processing unit) 30 executes FFT calculation with respect to each of the reception data strings 1-3. The FFT processing unit 30 performs frequency analysis of each of the reception data strings 1-3 over a plurality of time points, and obtains Doppler shift amounts (Doppler shift frequencies) over a plurality of time points.

FIG. 5 shows, for each of the time points, a Doppler shift amount (indicated by “◯”) obtained from the reception data string 1, a Doppler shift amount (indicated by “Δ”) obtained from the reception data string 2, and a Doppler shift amount (indicated by “□”) obtained from the reception data string 3.

The Doppler waveform generating unit 40 generates a Doppler waveform based on the Doppler shift amounts obtained for the respective reception data strings 1-3. The Doppler waveform generating unit 40 selects, for each time point, the maximum Doppler shift amount from among the reception data strings 1-3, and generates the Doppler waveform showing, across the plurality of time points, the maximum Doppler shift amounts corresponding to the respective time points.

FIG. 5 illustrates a specific example of the Doppler waveform generated by the Doppler waveform generating unit 40. In FIG. 5, the horizontal axis indicates time, while the vertical axis indicates velocity of the measurement target. The flow velocity range (+Vmax and −Vmax) is set by the user, for example.

The Doppler waveform generating unit 40 selects, for each time point, a Doppler shift amount having the maximum absolute value, for example, from among the Doppler shift amounts of the reception data strings 1-3. Further, the maximum Doppler shift amounts, which are obtained for the respective time points and successively across the plurality of time points, are connected using straight lines or curves, and the Doppler waveform is thereby formed.

The ultrasonic diagnostic device of FIG. 1 generates the Doppler waveform based on the reception data strings obtained from the plurality of measurement lines while moving the ultrasonic beam in periodic manner within the measurement area. Preferably, the intervals and the number of the plurality of measurement lines are set such that the range of movement of the measurement target is covered. By configuring as such, even in cases in which there is movement in the position of the blood flow (which serves as the measurement target, for example) at the location of the heart valve, the Doppler shift amounts can be measured from the rapid blood flow at the location of the heart valve by means of any of the plurality of measurement lines, and the Doppler waveform of the rapid blood flow at the location of the heart valve can be obtained.

It is noted that, based on the frequency spectrum data obtained for the respective reception data strings 1-3 by performing the FFT calculation, the Doppler waveform generating unit 40 may select, for each time point, a data string having the maximum frequency spectrum data intensity (i.e., highest sensitivity) from among the reception data strings 1-3, and thereby form the Doppler waveform.

While a preferred embodiment of the present invention has been explained above, the above-described embodiment simply represents one example in all its aspects, and does not serve to limit the scope of the present invention. The present invention includes various modifications within a scope that does not deviate from the principle of the invention.

### LIST OF REFERENCE SYMBOLS

10 probe; 12 transmission circuit; 14 transmission beam former; 16 reception circuit; 18 reception beam former; 20 sampling processing unit; 22 memory; 30 FFT processing unit; 40 Doppler waveform generating unit; 42 display unit.

1. An ultrasonic diagnostic device, comprising:
a probe that transmits and receives continuous ultrasonic waves;
a transmission-reception control unit that controls the probe to thereby acquire an ultrasonic reception signal while moving a continuous-wave ultrasonic beam in periodic manner;
a sampling processing unit that employs a plurality of sampling sets that are shifted in time phase from each other, and performs a sampling process on the reception signal over a plurality of time phases for each of the sampling sets, so as to obtain a reception data string for each of the sampling sets; and
a Doppler processing unit that obtains Doppler information based on a plurality of reception data strings corresponding to the plurality of sampling sets.

2. The ultrasonic diagnostic device according to claim 1, wherein
the transmission-reception control unit moves the ultrasonic beam in periodic manner so as to acquire the reception signal repeatedly at a plurality of measurement lines; and
the sampling processing unit employs the plurality of sampling sets corresponding to the plurality of measurement lines, and obtains, for each sampling set, a reception data string regarding its corresponding measurement line, thereby obtaining a plurality of reception data strings corresponding to the plurality of measurement lines.

3. The ultrasonic diagnostic device according to claim 2, wherein
the transmission-reception control unit employs a beam cursor set according to a user operation as a reference measurement line, sets an additional measurement line near the reference measurement line, and moves the ultrasonic beam in a periodic manner so as to acquire the reception signal repeatedly at the reference measurement line and the additional measurement line.

4. The ultrasonic diagnostic device according to claim 1, wherein
the Doppler processing unit analyzes a Doppler shift amount concerning each of the reception data strings, and generates a Doppler waveform based on a plurality of Doppler shift amounts obtained from the plurality of reception data strings.

5. The ultrasonic diagnostic device according to claim 4, wherein
the Doppler processing unit selects a maximum Doppler shift amount for each time point from among the plurality of reception data strings, and generates a Doppler waveform showing, across a plurality of time points, maximum Doppler shift amounts corresponding to respective ones of the plurality of time points.

6. The ultrasonic diagnostic device according to claim 1, wherein
the transmission-reception control unit causes the ultrasonic beam passing through a measurement area to be moved in a periodic manner within the measurement area, and, while doing so, acquires the reception signal along the ultrasonic beam.

7. The ultrasonic diagnostic device according to claim 6, wherein
the sampling processing unit performs a sampling process on the reception signal acquired while the ultrasonic beam is moved in a periodic manner within the measurement area, and thereby obtains the reception data string for each of the sampling sets.

8. The ultrasonic diagnostic device according to claim 7, wherein
the Doppler processing unit obtains the Doppler information in the measurement area based on the plurality of reception data strings corresponding to the plurality of sampling sets.

9. The ultrasonic diagnostic device according to claim 8, wherein
the Doppler processing unit analyzes a Doppler shift amount concerning each of the reception data strings, and generates a Doppler waveform based on a plurality of Doppler shift amounts obtained from the plurality of reception data strings.

10. The ultrasonic diagnostic device according to claim 9, wherein
the Doppler processing unit selects a maximum Doppler shift amount for each time point from among the plurality of reception data strings, and generates a Doppler waveform showing, across a plurality of time points, maximum Doppler shift amounts corresponding to respective ones of the plurality of time points.

11. The ultrasonic diagnostic device according to claim 6, wherein
the transmission-reception control unit moves the ultrasonic beam in periodic manner so as to acquire the reception signal repeatedly at a plurality of measurement lines passing through the measurement area.

12. The ultrasonic diagnostic device according to claim 11, wherein
the transmission-reception control unit employs a beam cursor set according to a user operation as a reference measurement line, sets an additional measurement line near the reference measurement line, and moves the ultrasonic beam in a periodic manner so as to acquire the reception signal repeatedly at the reference measurement line and the additional measurement line.

13. The ultrasonic diagnostic device according to claim 11, wherein
the sampling processing unit employs the plurality of sampling sets corresponding to the plurality of measurement lines, and obtains, for each sampling set, a reception data string regarding its corresponding measurement line, thereby obtaining a plurality of reception data strings corresponding to the plurality of measurement lines.

14. The ultrasonic diagnostic device according to claim 13, wherein
the Doppler processing unit obtains the Doppler information in the measurement area based on the plurality of reception data strings corresponding to the plurality of sampling sets.

15. The ultrasonic diagnostic device according to claim 14, wherein
the Doppler processing unit selects a maximum Doppler shift amount for each time point from among the plurality of reception data strings, and generates a Doppler waveform showing, across a plurality of time points, maximum Doppler shift amounts corresponding to respective ones of the plurality of time points.

16. An ultrasonic diagnostic device, comprising:
a probe that transmits and receives ultrasonic waves;
a transmission-reception control unit that controls the probe to thereby acquire an ultrasonic reception signal while moving an ultrasonic beam in a periodic manner;
a sampling processing unit that employs a plurality of sampling sets that are shifted in time phase from each other, and perform a sampling process on the reception signal over a plurality of time phases for each of the sampling sets, so as to obtain a reception data string for each of the sampling sets; and
a Doppler processing unit that obtains Doppler information based on a plurality of reception data strings corresponding to the plurality of sampling sets,
wherein the Doppler processing unit analyzes a Doppler shift amount concerning each of the reception data strings, and, in generating a Doppler waveform based on a plurality of Doppler shift amounts obtained from the plurality of reception data strings, the Doppler processing unit selects a maximum Doppler shift amount for each time point from among the plurality of reception data strings, and generates a Doppler waveform showing, across a plurality of time points, maximum Doppler shift amounts corresponding to respective ones of the plurality of time points.
